(19) United States
(12) Patent Application Publication
(10) Pub. No.: US 2009/0298936 A1
(43) Pub. Date: Dec. 3, 2009

(54) METHOD FOR ENERGIZING HUMAN BEINGS

(75) Inventors: James G. Clothier, JR., Ridgewood, NJ (US); Keith McDermott, Bound Brook, NJ (US); Patrice Bellon, Issy Les Moulineaux (FR)

Correspondence Address: CONNOLLY BOVE LODGE & HUTZ LLP, 1875 EYE STREET, N.W., SUITE 1100, WASHINGTON, DC, 20006, US

(73) Assignee: SYMRISE GmbH & Co. KG, Holzminden (DE)

(21) Appl. No.: 12/128,808

(22) Filed: May 29, 2008

(51) Int. Cl. A61K 31/215; A61K 31/01; A61K 31/045; A61K 31/22; A61P 43/00

(52) U.S. Cl. 514/530; 514/762; 514/729; 514/715; 514/547

(57) ABSTRACT

The present invention relates to a method for energizing a human being and to administering an energizing (personal care) composition to human beings wherein said composition contains one or more fragrance materials of group (a) of the present invention.

### FIELD OF THE INVENTION

The present invention relates to a method for energizing a human being and to administering an energizing (personal care) composition to human beings wherein said composition contains one or more fragrance materials of group (a) of the present invention.

### BACKGROUND OF THE INVENTION

Several currently marketed fragrant cosmetic products claim to have an “energizing” or “vitalizing” benefit to the user. Typically, these products possess fragrances that are purported to deliver these benefits. To support these claims, several methods have been employed to measure the effects of fragrance on physiological parameters with varying degrees of success, and unfortunately, much of the evidence for these purported benefits is the subject of folklore, rather than science.

It would be desirable to find other fragrance compositions that are capable of energizing, vitalizing and activating people.

### SUMMARY OF THE INVENTION

This invention relates to a method of energizing a human comprising administering to said human a personal care composition which comprises an effective amount of a fragrance material according to group (a) of the present invention, wherein the personal care composition is capable of energizing said human in terms of an increase of its heart rate (as determined per heartbeat measurement in beats per minute ((bpm)) by about 5% to about 100% based on the heart rate of said human being before administration of said amount of fragrance material.

Various ways to measure the heartbeat of a human being are known in the art, preferably the heartbeat is measured by a device generally known as a heart rate monitor, which allows to measure the heart rate of a human being in real time.

Data collection and analysis of each individual's heart rate is preferably performed using a heart rate sensor, preferably a finger clip (i.e. finger clip attachment for heartbeat measurement) or an ear sensor, more preferably using the Heart Rhythm Monitor using Freeze-Framer® 2.0 software (now available as emWave® pc) from HeartMath®, 14700 West Park Avenue, Boulder Creek, Calif.

For example, U.S. Pat. No. 6,358,201 and U.S. Pat. No. 7,163,512 describe a method for quantifying and analyzing heart rhythms which is called analysis of heart rate variability (HRV). Heart rate variability, which is derived from the electrocardiogram or pulse, is a measure of these naturally occurring beat-to-beat changes in heart rate and is an important indicator of health and fitness. HRV is influenced by a variety of factors, including physical movement, sleep and mental activity, and is particularly responsive to stress and changes in emotional state.

A primary aspect of the present invention generally relates to a method of energizing a human being, the method comprising administering to said human being a personal care composition which comprises an effective amount of one or more energizing fragrance materials selected from group (a) of the present invention consisting of:

(a) tetrahydrogeraniol, methyl dihydro jasmonate (Hedione®), 1-menthol, 1,1-dimethoxy-2,2,5-trimethyl-4-hexene (Amarocite®), n-decanal (aldehyde C10), diethyl malonate and anethol.

More particularly, the present invention relates to a method of energizing a human being comprising the following steps:
providing an effective amount of a fragrance material comprising or consisting of one or more compounds selected from group (a) consisting of:

(a) tetrahydrogeraniol, methyl dihydro jasmonate, I-menthol, 1,1-dimethoxy-2,2,5-trimethyl-4-hexene, n-decanal, diethyl malonate, and anethol, and
administering said effective amount of fragrance material to said human being in a way that said amount of fragrance material reaches the nasal cavity of said human being,

wherein said administered amount of fragrance material is capable of increasing the heart rate of said human being, preferably by 5% to 100%, more preferably by 5 to 50%, most preferably by 10 to 25%, based on the heart rate of said human being before administration of said amount of fragrance material.

The invention likewise relates to the use of a fragrance material comprising one or more compounds selected from the group (a) consisting of tetrahydrogeraniol, methyl dihydro jasmonate, I-menthol, 1,1-dimethoxy-2,2,5-trimethyl-4-hexene, n-decanal, diethyl malonate, and anethol.

### DETAILED DESCRIPTION OF THE INVENTION

This invention relates to a method of energizing humans beings, in particular as defined above and in the attached set of claims. Preferably the one or more compounds of group (a) are selected from the group consisting of:

tetrahydrogeraniol, methyl dihydro jasmonate, I-menthol, 1,1-dimethoxy-2,2,5-trimethyl-4-hexene, a mixture of n-decanal and tetrahydrogeraniol, a mixture of n-decanal and I-menthol, a mixture of I-menthol and diethyl malonate, a mixture of 1,1-dimethoxy-2,2,5-trimethyl-4-hexene and anethol.

If mixtures of two fragrance materials of (preferred) group (a) are selected, the ratio by weight of these two materials is preferably in the range of from 5:1 to 1:5, more preferably in the range of from 3:1 to 1:3, most preferably in the range of from 2:1 to 1:2.

In the context of the present invention, the term “administering” refers to fragrances or sensory complexes according to the present invention to be perceived (i) through the nose (orthonasally) or (ii) through the mouth (retronasally), i.e. through the oral cavity, or both.

Thus, the term “administering” includes (i) inhalation of a topically applied personal care composition; (ii) inhalation of the vapors which are released when a personal care composition is dissolved or dispersed in a liquid vehicle such as water, (iii) inhalation of vapors which are released when a personal care composition is dispersed, sprayed, melted or burned, or (iv) retronasal perception of vapors in case the personal care composition is placed into the human oral cavity.

The term “energizing” as used herein refers to invigoration, activation, stimulation, vivication, vitalization or enlivenment of a human being, preferably of an adult having an age from 18 to about 75 years. In particular, a human being is energized when its heart rate is increased by 5% or more.

The term “effective amount” refers to the amount of the fragrance materials according to group (a) of the present invention which is needed to create the desired energizing response in a human being, and in particular an adult human being.

Another aspect of the present invention includes a “sensory complex” capable of energizing a human. In a preferred method (or use) of the present invention one or more compounds selected from said group (a), preferably said effective amount of said fragrance material comprising one or more compounds selected from said group (a), are part of a sensory complex, wherein the sensory complex comprises or consists of:

(i) an effective amount of a fragrance material comprising one or more compounds selected from the group (a) of the present invention, and additionally

(ii) one or more fragrance compounds different from the compounds of group (a),

(iii) one or more essential oils, and/or

(iv) one or more cosmetically acceptable diluents.

Further preferred is a method (and a use), wherein the sensory complex comprises or consists of:

(i) a total amount of from 1% to 80% by weight of one or more compounds selected from group (a) of the present invention, and additionally

(ii) a total amount of from 0.5% to 95% by weight of one of more fragrance compounds different from the compounds of group (a),

and/or

(iii) a total amount of from 0.01% to 30% by weight of one or more essential oils,

and/or

(iv) a total amount of from 1% to 80% by weight of one or more cosmetically acceptable diluents,

in each case based on the total weight of the sensory complex.

Typically, the total amount of compounds of group (a) of the sensory complex is from about 1% to about 80%, preferably about 2% to about 70%, more preferably about 4% to about 65%, more preferably about 5% to about 60%, based upon the total weight of the sensory complex.

Typically, the total amount of one or more fragrance compounds (ii) of the sensory complex, preferably selected from group (b) as illustrated below, is from about 0.5% to about 95%, preferably from about 1% to about 75%, based upon the total weight of the sensory complex.

Typically, the total amount of the essential oil portion (iii) of the sensory complex, preferably selected from group (c) as illustrated below, is from about 0.01% to about 30%, preferably from about 0.1% to about 10%, based upon the total weight of the sensory complex.

Typically, the total amount of cosmetically acceptable diluents (iv) of the sensory complex, preferably selected from group (d) as illustrated below, is from about 1% to about 80%, preferably from about 5% to about 50%, based upon the total weight of the sensory complex.

In a particulary preferred embodiment a “sensory complex” as used in the methods of the present invention consists of (i) one or more compounds of group (a) of the present invention, (ii) one or more fragrance compounds different from the compounds of group (a), selected from group (b), (iii) one or more essential oils, selected from group (c), and (iv) one or more cosmetically acceptable diluents, selected from group (d), wherein the amounts of groups (a), (b), (c) and (d) are in the above given (preferred) weight percentage ranges and the sum of the amounts of groups (a), (b), (c) and (d) is 100%.

The fragrance compounds of the sensory complex are preferably selected from a group (b) consisting of:
Hydrocarbons, preferably selected from the group consisting of 3-carene; α-pinene; β-pinene; α-terpinene; γ-terpinene; p-cymene; bisabolene; camphene; caryophyllene, cedrene; farnesene; liminene; longifolene; myrcene; ocimene; valencene; (E,Z)-1,3,5-undecatriene;
Aliphatic alcohols, preferably selected from the group consisting of hexanol; octanol; 3-octanol; 2,6-dimethylheptanol; 2-methylheptanol; 2-methyloctanol; (E)-3-hexenol; (E) and (Z)-3-hexenol; 1-octen-3-ol; mixtures of 3,4,5,6,6-pentamethyl-3/4-hepten-2-ol and 3,5,6,6-tetramethyl-4-methyleneheptan-2-ol; (E,Z)-2,6-nonadienol; 3,7-dimethyl-7-methoxyoctan-2-ol; 9-decenol; 10-undecenol; 4-methyl-3-decen-5-ol;
Alphatic aldehydes and their acetals, preferably selected from the group consisting of heptanal; octanal; nonanal; undecanal; dodecanal; tridecanal; 2-methyloctanal; 2-methylnonanal; (E)-2-hexenal; (Z)-4-heptenal; 2,6-dimethyl-5-heptenal; 10-undecenal; (E)-4-decenal; 2-dodecenal; 2,6,10-trimethyl-5,9-undecadienal; heptanal diethyl acetal; citronellyl oxyacetaldehyde;
Aliphatic ketones and oximes thereof, preferably selected from the group consisting of 2-heptanone; 2-octanone; 3-octanone; 2-nonanone; 5-methyl-3-heptanone; 5-methyl-3-heptanone oxime; 2,4,4,7-tetramethyl-6-octen-3-one;
Aliphatic sulphur-containing compounds, preferably selected from the group consisting of 3-methylthiohexanol; 3-methylthiohexyl acetate; 3-mercaptohexanol; 3-mercaptohexyl acetate; 3-mercaptohexyl butyrate; 3-acetylthiohexyl acetate; 1-menthene-8-thiol;
Aliphatic nitriles, preferably selected from the group consisting of 2-nonenenitrile; 2-tridecenenenitrile; 2,12-tridecenene-nitrile; 3,7-dimethyl-2,6-octadienenitrile; 3,7-dimethyl-6-octenenitrile;
Aliphatic carboxylic acids and esters thereof, preferably selected from the group consisting of (E)- and (Z)-3-hexenyl formate; ethyl acetoacetate; isoamyl acetate; hexyl acetate; 3,5,5-trimethylhexyl acetate; 3-methyl-2-butenyl acetate; (E)-2-hexenyl acetate; (E)- and (Z)-3-hexenyl acetate; octyl acetate; 3-octyl acetate; 1-octen-3-yl acetate; ethyl butyrate; butyl butyrate; isoamyl butyrate; hexyl butyrate; (E)- and (Z)-3-hexenyl isobutyrate; hexyl crotonate; ethyl isovalerate; ethyl 2-methylpentanoate; ethyl hexanoate; allyl hexanoate; ethyl heptanoate; allyl heptanoate; ethyl octanoate; ethyl (E,Z)-2,4-decadienoate; methyl 2-octynoate; methyl 2-nonynoate; allyl-2-isoamyloxyacetate; methyl-3,7-dimethyl-2,6-octadienoate;
Acyclic terpene alcohols, preferably selected from the group consisting of citronellol; geraniol; nerol; linalool; lavandulol; nerolidol; farnesol; tetrahydrolinalool; 2,6-dimethyl-7-octen-2-ol; 2,6-dimethyloctan-2-ol; 2-methyl-6-methylene-7-octen-2-ol; 2,6-dimethyl-5,7-octadien-2-ol; 2,6-dimethyl-3,5-octadien-2-ol; 3,7-dimethyl-4,6-octadien-3-ol; 3,7-dimethyl-1,5,7-octatrien-3-ol; 2,6-dimethyl-2,5,7-octatrien-1-ol; and formates, acetates, propionates, isobutyrates, butyrates, isovalerates, pentanoates, hexanoates, crotonates, tiglinates, 3-methyl-2-butenoates thereof;
Acyclic terpene aldehydes and ketones, preferably selected from the group consisting of geranial; neral; citronellal; 7-hydroxy-3,7-dimethyloctanal; 7-methoxy-3,7-dimethyloctanal; 2,6,10-trimethyl-9-undecenal; geranylacetone; and the dimethyl and diethyl acetals of geranial, neral, 7-hydroxy-3,7-dimethyloctanal;
Cyclic terpene alcohols, preferably selected from the group consisting of isopulegol; alpha-terpineol; terpineol-4; menthan-8-ol; menthan-1-ol; menthan-7-ol; borneol; isoborneol; linalool oxide; nopol; cedrol; ambrinol; vetiverol; guaiol; and the formates, acetates, propionates, isobutyrates, butyrates, isovalerates, pentanoates, hexanoates, crotonates, tiglinates, 3-methyl-2-butenoates thereof;
Cyclic terpene aldehydes and ketones, preferably selected from the group consisting of menthone; isomenthone; 8-mercaptomenthan-3-one; carvone; camphor; fenchone; alpha-ionone; beta-ionone; alpha-n-methylionone; beta-n-methylionone; alpha-isomethylionone; beta-isomethylionone; alpha-irone; alpha-damascone; beta-damascone; beta-damascenone; delta-damascone; gamma-damascone; 1-(2,4,4-trimethyl-2-cyclohexen-1-yl)-2-buten-1-one; 1,3,4,6,7,8a-hexahydro-1,1,5,5-tetramethyl-2H-2,4a-methanonaphthalen-8(5H)-one; nootkatone; dihydronootkatone; alpha-sinensal; beta-sinensal; acetylated cedarwood oil (methyl cedryl ketone);
Cyclic alcohols, preferably selected from the group consisting of 4-tert.-butylcyclohexanol; 3,3,5-trimethylcyclohexanol; 3-isocamphylcyclohexanol; 2,6,9-trimethyl-Z2,Z5,E9-cyclododecatrien-1-ol; 2-isobutyl-4-methyltetrahydro-2H-pyran-4-ol;
Cycloaliphatic alcohols, preferably selected from the group consisting of alpha-3,3-trimethylcyclohexylmethanol; 2-methyl-4-(2,2,3-trimethyl-3-cyclopent-1-yl)butanol; 2-methyl-4-(2,2,3-trimethyl-3-cyclopent-1-yl)-2-buten-1-ol; 2-ethyl-4-(2,2,3-trimethyl-3-cyclopent-1-yl)-2-buten-1-ol; 3-methyl-5-(2,2,3-trimethyl-3-cyclopent-1-yl)-pentan-2-ol; 3-methyl-5-(2,2,3-trimethyl-3-cyclopent-1-yl)-4-penten-2-ol; 3,3-dimethyl-5-(2,2,3-trimethyl-3-cyclopent-1-yl)-4-penten-2-ol; 1-(2,2,6-trimethylcyclohexyl)pentan-3-ol; 1-(2,2,6-trimethylcyclohexyl)hexan-3-ol;
Cyclic and cycloaliphatic ethers, preferably selected from the group consisting of cineol; cedryl methyl ether; cyclododecyl methyl ether; (ethoxymethoxy)cyclododecane; alpha-cedrene epoxide; 3a,6,6,9a-tetramethyldodecahydronaphtho[2,1-b]furan; 3a-ethyl-6,6,9a-trimethyldodecahydronaphtho[2,1-b]furan; 1,5,9-trimethyl-13-oxabicyclo[10.1.0]trideca-4,8-diene; rose oxide; 2-(2,4-dimethyl-3-cyclohexen-1-yl)-5-methyl-5-(1-methylpropyl)-1,3-dioxane;
Cyclic ketones, preferably selected from the group consisting of 4-tert.-butylcyclohexanone; 2,2,5-trimethyl-5-pentylcyclopentanone; 2-heptylcyclopentanone; 2-pentylcyclopentanone; 2-hydroxy-3-methyl-2-cyclopenten-1-one; 3-methyl-cis-2-penten-1-yl-2-cyclopenten-1-one; 3-methyl-2-pentyl-2-cyclopenten-1-one; 3-methyl-4-cyclopentadecenone; 3-methyl-5-cyclopentadecenone; 3-methylcyclopentadecanone; 4-(1-ethoxyvinyl)-3,3,5,5-tetramethylcyclohexanone; 4-tert.-pentylcyclohexanone; 5-cyclohexadecen-1-one; 6,7-dihydro-1,1,2,3,3-pentamethyl-4(5H)-indanone; 9-cycloheptadecen-1-one; cyclopentadecanone; cyclohexadecanone;
Cycloaliphatic aldehydes, preferably selected from the group consisting of 2,4-dimethyl-3-cyclohexenecarbaldehyde; 2-methyl-4-(2,2,6-trimethyl-cyclohexen-1-yl)-2-butenal; 4-(4-hydroxy-4-methylpentyl)-3-cyclohexenecarbaldehyde; 4-(4-methyl-3-penten-1-yl)-3-cyclohexenecarbaldehyde;
Cycloaliphatic ketones, preferably selected from the group consisting of 1-(3,3-dimethylcyclohexyl)-4-penten-1-one; 1-(5,5-dimethyl-1-cyclohexen-1-yl)-4-penten-1-one; 2,3,8,8-tetramethyl-1,2,3,4,5,6,7,8-octahydro-2-naphthalenyl methyl ketone; methyl-2,6,10-trimethyl-2,5,9-cyclododecatrienyl ketone; tert.-butyl (2,4-dimethyl-3-cyclohexen-1-yl) ketone;
Esters of cyclic alcohols, preferably selected from the group consisting of 2-tert.-butylcyclohexyl acetate; 4-tert.-butyl-cyclohexyl acetate; 2-tert.-pentylcyclohexyl acetate; 4-tert.-pentylcyclohexyl acetate; decahydro-2-naphthyl acetate; 3-pentyltetrahydro-2H-pyran-4-yl acetate; decahydro-2,5,5,8a-tetramethyl-2-naphthyl acetate; 4,7-methano-3a,4,5,6,7,7a-hexahydro-5 or 6-indenyl acetate; 4,7-methano-3a,4,5,6,7,7a-hexahydro-5 or 6-indenyl propionate; 4,7-methano-3a,4,5,6,7,7a-hexahydro-5 or 6-indenyl isobutyrate; 4,7-methanooctahydro-5 or 6-indenyl acetate;
Esters of cycloaliphatic carboxylic acids, preferably selected from the group consisting of, allyl 3-cyclohexylpropionate; allyl cyclohexyloxyacetate; methyl jasmonate; methyl 2-hexyl-3-oxocyclopentanecarboxylate; ethyl 2-ethyl-6,6-dimethyl-2-cyclohexenecarboxylate; ethyl 2,3,6,6-tetramethyl-2-cyclohexenecarboxylate; ethyl 2-methyl-1,3-dioxolane-2-acetate;
Aromatic hydrocarbons, preferably selected from the group consisting of styrene and diphenylmethane;
Araliphatic alcohols, preferably selected from the group consisting of benzyl alcohol; 1-phenylethyl alcohol; 2-phenylethyl alcohol; 3-phenylpropanol; 2-phenylpropanol; 2-phenoxyethanol; 2,2-dimethyl-3-phenylpropanol; 2,2-dimethyl-3-(3-methylphenyl)propanol; 1,1-dimethyl-2-phenylethyl alcohol; 1,1-dimethyl-3-phenylpropanol; 1-ethyl-1-methyl-3-phenylpropanol; 2-methyl-5-phenylpentanol; 3-methyl-5-phenylpentanol; 3-phenyl-2-propen-1-ol; 4-methoxybenzyl alcohol; 1-(4-isopropylphenyl)ethanol;
Esters of araliphatic alcohols and aliphatic carboxylic acids, preferably selected from the group consisting of benzyl propionate; benzyl isobutyrate; benzyl isovalerate; 2-phenylethyl acetate; 2-phenylethyl propionate; 2-phenylethyl isobutyrate; 2-phenylethyl isovalerate; 1-phenylethyl acetate; alpha-trichloromethylbenzyl acetate; alpha,alpha-dimethylphenylethyl acetate; alpha,alpha-dimethylphenylethyl butyrate; cinnamyl acetate; 2-phenoxyethyl isobutyrate; 4-methoxybenzyl acetate;
Araliphatic ethers, preferably selected from the group consisting of 2-phenylethyl methyl ether; 2-phenylethyl isoamyl ether; 2-phenylethyl 1-ethoxyethyl ether; phenylacetaldehyde dimethyl acetal; phenylacetaldehyde diethyl acetal; hydratropaldehyde dimethyl acetal; phenylacetaldehyde glycerol acetal; 2,4,6-trimethyl-4-phenyl-1,3-dioxane; 4,4a,5,9b-tetrahydroindeno[1,2-d]-m-dioxin; 4,4a,5,9b-tetrahydro-2,4-dimethylindeno[1,2-d]-m-dioxin;
Aromatic and araliphatic aldehydes, preferably selected from the group consisting of benzaldehyde; phenylacetaldehyde; 3-phenylpropanal; hydratropaldehyde; 4-methylbenzaldehyde; 4-methylphenylacetaldehyde; 3-(4-ethylphenyl)-2,2-dimethylpropanal; 2-methyl-3-(4-isopropylphenyl)propanal; 2-methyl-3-(4-tert.-butylphenyl)propanal; 3-(4-tert.-butylphenyl)propanal; cinnamaldehyde; alpha-butylcinnamaldehyde; alpha-amylcinnamaldehyde; alpha-hexylcinnamaldehyde; 3-methyl-5-phenylpentanal; 4-methoxybenzaldehyde; 4-hydroxy-3-methoxybenzaldehyde; 4-hydroxy-3-ethoxybenzaldehyde; 3,4-methylenedioxybenzaldehyde; 3,4-dimethoxy-benzaldehyde; 2-methyl-3-(4-methoxyphenyl)propanal; 2-methyl-3-(4-methylenedioxyphenyl)propanal;
Aromatic and araliphatic ketones, preferably selected from the group consisting of acetophenone; 4-methylacetophenone; 4-methoxyacetophenone; 4-tert.-butyl-2,6-dimethylacetophenone; 4-phenyl-2-butanone; 4-(4-hydroxyphenyl)-2-butanone; 1-(2-naphthalenyl)ethanone; benzophenone; 1,1,2,3,3,6-hexamethyl-5-indanyl methyl ketone; 6-tert.-butyl-1,1-dimethyl-4-indanyl methyl ketone; 1-[2,3-dihydro-1,1,2,6-tetramethyl-3-(1-methylethyl)-1H-5-indenyl]ethanone; 5′,6′,7′,8′-tetrahydro-3′,5′,5′,6′,8′,8′-hexamethyl-2-acetonaphthone;
Aromatic and araliphatic carboxylic acids and esters thereof, preferably selected from the group consisting of benzoic acid; phenylacetic acid; methyl benzoate; ethyl benzoate; hexyl benzoate; methyl phenylacetate; ethyl phenylacetate; geranyl phenylacetate; phenylethyl phenylacetate; methyl cinnamate; ethyl cinnamate; benzyl cinnamate; phenylethyl cinnamate; cinnamyl cinnamate; allyl phenoxyacetate; methyl salicylate; isoamyl salicylate; hexyl salicylate; cyclohexyl salicylate; cis-3-hexenyl salicylate; benzyl salicylate; phenylethyl salicylate; methyl 2,4-dihydroxy-3,6-dimethylbenzoate; ethyl 3-phenylglycidate; ethyl 3-methyl-3-phenylglycidate;
Nitrogen-containing aromatic compounds, preferably selected from the group consisting of 2,4,6-trinitro-1,3-dimethyl-5-tert.-butylbenzene; 3,5-dinitro-2,6-dimethyl-4-tert.-butylacetophenone; cinnamonitrile; 5-phenyl-3-methyl-2-pentenenitrile; 5-phenyl-3-methylpentanenitrile; methyl anthranilate; methyl N-methylanthranilate; Schiff bases of methyl anthranilate with 7-hydroxy-3,7-dimethyloctanal; 2-methyl-3-(4-tert.-butylphenyl)propanal or 2,4-dimethyl-3-cyclohexene-carbaldehyde; 6-isopropylquinoline; 6-isobutylquinoline; 6-sec.-butylquinoline; indole; skatole; 2-methoxy-3-isopropylpyrazine; 2-isobutyl-3-methoxypyrazine;
Phenols, phenyl ethers and phenyl esters, preferably selected from the group consisting of estragole; eugenol; eugenyl methyl ether; isoeugenol; isoeugenyl methyl ether; thymol; carvacrol; diphenyl ether; beta-naphthyl methyl ether; beta-naphthyl ethyl ether; beta-naphthyl isobutyl ether; 1,4-dimethoxybenzene; eugenyl acetate; 2-methoxy-4-methylphenol; 2-ethoxy-5-(1-propenyl)phenol; p-cresyl phenylacetate;
Heterocyclic compounds, preferably selected from the group consisting of 2,5-dimethyl-4-hydroxy-2H-furan-3-one; 2-ethyl-4-hydroxy-5-methyl-2H-furan-3-one; 3-hydroxy-2-methyl-4H-pyran-4-one; 2-ethyl-3-hydroxy-4H-pyran-4-one;
Lactones, preferably selected from the group consisting of 1,4-octanolide; 3-methyl-1,4-octanolide; 1,4-nonanolide; 1,4-decanolide; 8-decen-1,4-olide; 1,4-undecanolide; 1,4-dodecanolide; 1,5-decanolide; 1,5-dodecanolide; 1,15-pentadecanolide; cis and trans-11-pentadecen-1,15-olide; cis and trans-12-pentadecen-1,15-olide; 1,16-hexadecanolide; 9-hexadecen-1,16-olide; 10-oxa-1,16-hexadecanolide; 11-oxa-1,16-hexadecanolide; 12-oxa-1,16-hexadecanolide; ethylene 1,12-dodecanedioate; ethylene 1,13-tridecanedioate; coumarin; 2,3-dihydrocoumarin; octahydrocoumarin.

More preferred fragrance compounds of group (b) of the sensory complex are selected from the group consisting of:

AGRUMEX ®
AGRUNITRIL
ALDEHYDE C8
ALDEHYDE C9
ALDEHYDE C10
ALDEHYDE C11 UNDECYLENIC
ALDEHYDE C12 LAURIC
ALDEHYDE C14 SO-CALLED
ALLYL AMYL GLYCOLATE
ALLYL CAPROATE
ALLYL CYCLOHEXYL PROPIONATE
ALLYL HEPTOATE
ALLYL IONONE
AMBROXAN ®
ANETHOL
ANISYL ACETATE
BENZALDEHYDE
BOURGEONAL ®
CARVONE L
DAMASCENONE
DAMASCONE DELTA
DIHYDROEUGENOL
DIHYDROMYRCENOL
DIMETHYL BENZYL CARBINYL ACETATE
DIMETHYL BENZYL CARBINYL BUTYRAT
ETHYL BUTYRATE
ETHYL CAPROATE
ETHYL LINALOOL
ETHYL MALTOL
ETHYL METHYL BUTYRATE-2
ETHYL VANILLIN
ETHYLENE BRASSYLATE
EUCALYPTOL
EVERNYL ®
FLORAZON ®
GERANIOL
GLOBALIDE ®
HERBAFLORAT ®
HERBYL PROPIONATE
HEXENOL CIS-3
HEXENYL ACETATE CIS-3
HEXYL ACETATE
INDOLE
IONONE BETA
ISO E SUPER ®
JASMOPYRANE ®
LIGUSTRAL ®
LILIAL ®
LINALOOL OXIDE
LINALYL ACETATE
LINALYL ISOBUTYRATE
MACROLIDE ®
MALTOL
MELONAL ®
MENTHONE L
ISOMENTHONE
MENTHYL ACETATE
METHYL ANTHRANILATE
METHYL HEPTENONE-6,5,2
MUGETANOL ®
PHENIRAT ®
PHENOXANOL ®
PHENYLETHYL ACETATE
PHENYLETHYL ALCOHOL
PHENYLETHYL ALCOHOL
PHENYLETHYL PHENYLACETATE
PRENYL ACETATE
ROSE OXIDE
TETRAHYDROLINALOOL
THYMOL
UNDECAVERTOL ®
VANILLIN

The corresponding chemical names and/or structures of the preferred fragrance compounds of group (b) can be found in H. Surburg and J. Panten, Common Fragrance and Flavor Materials, 5^{th} edition, Wiley-VCH, Weinheim 2006.

The essential oils of the sensory complex are preferably selected from group (c) consisting of:

amyris oil; angelica seed oil; angelica root oil; aniseed oil; valerian oil; basil oil; bay oil; mugwort oil; benzoin resin; bergamot oil; birch tar oil; bitter almond oil; savory oil; bucco-leaf oil; cabreuva oil; cade oil; calamus oil; camphor oil; cananga oil; cardamom oil; cascarilla oil; cassia oil; cedar-leaf oil; cedarwood oil; cistus oil; citronella oil; lemon oil; coriander oil; costus root oil; cumin oil; cypress oil; davana oil; dill oil; dillseed oil; eucalyptus citriodora oil; eucalyptus oil; fennel oil; fir oil; galbanum oil; geranium oil; grapefruit oil; guaiac wood oil; ginger oil; calamus oil; blue chamomile oil; Roman chamomile oil; carrot-seed oil; cascarilla oil; pine-needle oil; spearmint oil; caraway oil; labdanum oil; lavandin oil; lavender oil; lemongrass oil; lovage oil; distilled lime oil; pressed lime oil; linaloe oil; litsea cubeba oil; bay-leaf oil; mace oil; marjoram oil; mandarin oil; massoi bark oil; ambrette oil; clary sage oil; myristica oil; myrrh oil; myrtle oil; clove leaf oil; clove flower oil; neroli oil; olibanum oil; opopanax oil; orange oil; origanum oil; palma-rosa oil; patchouli oil; perilla oil; Peru balsam oil; parsley leaf oil; parsley seed oil; petitgrain oil; peppermint oil; pepper oil; pimento oil; pine oil; pennyroyal oil; rosewood oil; rose oil; rosemary oil; Dalmatian sage oil; Spanish sage oil; sandalwood oil; celery seed oil; spike lavender oil; Japanese aniseed oil; styrax oil; tagetes oil; fir-needle oil; tea-tree oil; turpentine oil; thyme oil; verbena oil; vetiver oil; juniper oil; wormwood oil; wintergreen oil; ylang oil; hyssop oil; cinnamon leaf oil; cinnamon bark oil.

The essential oils of group (c) of the sensory complex are more preferably selected from the group consisting of: cardamom oil, chamomile oil, cinnamon oil, clove oil, geranium oil, ginger oil, grapefruit oil, lemon oil, lemongrass oil, orange oil, peppermint oil, pine oil, rosemary oil, spearmint oil, thyme oil.

The cosmetically acceptable diluents of the sensory complex are preferably selected from group (d) consisting of ethanol, dipropylene glycol, propylene glycol, 1,2-butylene glycol, glycerol, diethylene glycol monoethyl ether, diethyl phthalate, isopropyl myristate, triethyl citrate, benzyl benzoate and benzyl acetate.

In preferred methods (and uses) of the present invention the effective amount of the fragrance material comprising one or more compounds selected from said group (a) is part of a personal care composition.

In preferred methods (or uses) of the present invention a sensory complex is used (as defined above), the sensory complex preferably being part of a personal care composition.

In preferred methods of the present invention, when the effective amount of fragrance material above is part of a personal care composition, said effective amount of said fragrance material comprising one or more compounds selected from said group (a) is in the range of from 0.01 to 2%, preferably in the range of from 0.05 to 1%, based on the total weight of the personal care composition.

In further preferred methods of the present invention, when a sensory complex as described above is part of a personal care composition, the total amount of the sensory complex is in the range of from 0.05 to 10%, preferably in the range of from 0.2 to 2%, based on the total weight of the personal care composition.

The sensory complex used in the methods of the present invention may be produced by blending

(i) one or more compounds of group (a) of the present invention, and

(ii) one or more fragrance compounds different from the compounds of group (a), preferably selected from group (b),

(iii) one or more essential oils, selected from group (c), and/or

(iv) one or more cosmetically acceptable diluents, preferably selected form group (d)

under ambient conditions until the final mixture is homogenous, using equipment and methodology commonly known in the art of fragrance compounding. It is preferable to store the final sensory complex mixture under ambient conditions for a few hours after mixing before using it as a component of a personal care composition.

Personal care compositions used in preferred methods of the present invention may be produced by blending the desired components with the sensory complex using equipment and methodology commonly known in the art of personal care product manufacture. In order to improve the solubilization of the sensory complex in aqueous personal care compositions, the sensory complex may be preblended with one or more of the nonionic surfactants.

Personal care composition for use in the methods of the present invention refer to personal cosmetic, toiletry, and healthcare products and are preferably selected from the group consisting of leave-on products, rinse-off products, oral care products, oral care devices, air-care products or air-care devices,

such as wipes, washes, baths, shampoos, gels, soaps, sticks, balms, sachets, pillows, mousses, sprays, lotions, creams, cleansing compositions, powders, oils, bath oils and other bath compositions which may be added to a bath.

Personal care compositions may also include, but are not limited to, aerosols, candles, and substances that may be used with vaporizers. The aforementioned wipes, washes, baths, shampoos, gels, soaps, sticks, balms, sachets, pillows, mousses, sprays, lotions, creams, cleansing compositions, oils, bath oils, aerosols, candles and substances which may be used with vaporizers are commercially known to those who have a knowledge of preparing personal care compositions.

Preferred leave-on products for use in the methods of the invention are selected from the group consisting of wipes, gels, sticks, mousses, sprays, lotions, creams, facial masks, cleansing compositions, powders, and oils.

Preferred rinse-off products for use in the methods of the invention are selected from the group consisting of wipes, washes, baths, shampoos, gels, soaps, sticks, balms, sachets, pillows, mousses, sprays, lotions, creams, cleansing compositions, powders, oils, and bath oils.

Alternative preferred personal care composition for use in the methods of the invention are selected from the group consisting of air-care products or devices selected from the group consisting of pump-sprays, aerosols, candles, vaporizers, diffusers, wicking systems, fans, piezoelectric systems, heated oil baths, and incense sticks.

Alternative preferred personal care composition for use in the methods of the present invention are oral care products selected from the group consisting of toothpastes, dentifrices, mouth sprays, mouth washes, chewing gums, mouth cleansers, and dental devices.

Personal care compositions for use in the methods of the invention comprising one or more compounds of group (a) or a sensory complex as described above are capable of increasing the heart rate, preferably by 5% to 100%, more preferably by 5 to 50%, most preferably by 10 to 25%.

In addition, the invention includes a personal care composition comprising an effective amount of the sensory complex according to the present invention and a method of energizing, invigorating, activating, stimulating, vivicating, vitalizing or enliving a human being comprising administering to said human being a personal care composition comprising an effective amount of the sensory complex according to the present invention.

One method of energizing a human is via the use of a personal care composition as described above that comprises, based upon the total weight of the personal care composition, from 0.05 to 10%, preferably from 0.20% to 2.5%, more preferably from 0.2 to 2%, most preferably from 0.3% to 1.5%, by weight of a sensory complex according to the present invention.

In order to achieve the desired response in a human being, the personal care composition may be used in a dosing amount that is in accordance with the prescribed directions of the personal care composition.

The personal care composition may be massaged or placed onto the skin of a human being at any time in accordance with the use instructions of the personal care product. This allows the fragrance to be released from the product slowly and enter the nasal cavity of a human.

Further aspects of the present invention are described in the examples and the attached set of claims.

### EXAMPLES

In order to illustrate the invention the following examples are included. These examples do not limit the invention. Those knowledgeable in the art may find other methods of practicing the invention. Those methods are deemed to be within the scope of this invention.

Unless stated otherwise all figures, data and percentages refer to the weight.

### Example 1

### Results of the Energizing Fragrance Materials According to Group (a) of the Present Invention

In order to introduce compounds of group (a) or a sensory complex as described above into the nasal cavity that will occur using many types of products containing the sensory complex the following method of human testing and data collection was developed, standardized and used to collect and analyze data to substantiate the energizing effects of the sensory complex materials in group (a).

Data collection and analysis of each panelists's heart rate was made using a finger clip attachment for the Heart Rhythm Monitor using Freeze-Framer® 2.0 software from HeartMath®, 14700 West Park Avenue, Boulder Creek, Calif.

The test is divided into 3 parts:
1) the pre-sniff-test period: places the panelist in a neutral state (15 min),
2) the sniff-test period (5 min),
3) the post-sniff-test period (10 min). Typically during each test session, three fragrances (single compound or mixture of compounds) are presented to the panelist, so one test lasts for one hour.

The method used involved the following protocol:
The stimulating effects of the fragrances (single compound or mixture of compounds) tested on 40 healthy human subjects (panelists)
All subjects have to be non-smokers with a normal body mass index, who are free of allergies to odorants and do not receive any medical treatment at the time of testing
Male and female subjects aged between 18 and 35 years participated in the study
Subjects are tested in individual sessions.
All experiments are conducted in a bright and quiet room
The ambient temperature should be between 24-26° C.
Upon arrival, the subject is identified through his personal data: name, age, sex and body mass index
In addition, he is asked about his mental and emotional condition (no persons under medical anti-depressant prescription are permitted).
Subsequently, panelists are informed about the proceedings
They are informed that a fragrance will be presented, but are unaware of the kind of fragrance and the exact time of administration
At 48 hours prior to testing, panelists had to abstain from food, beverages and toiletries containing tea, coffee and alcohol
Panelists are seated in a semi-reclined position, providing easy access to attach the finger sensor
During the testing, the panelist mustn't move nor talk
Panelist can close his or her eyes, think about pleasant things or breathe in and out deeply
After completion of the interview and rating scales, the heartbeat measurement can start
A fragrance (single compound or mixture of compounds) or a placebo substance is administered
Fragrances are diluted in an odorless solvent (e.g. mineral oil) which can also be used as placebo substance
Dilution of the fragrance: a total of 0.3 wt.-% to 2 wt.-% according to the odor intensity of the fragrance
The heartbeat before, during and after the inhalation of the fragrance for each panelist is monitored
Immediately after each test, the panelist will rate the pleasantness of the fragrance through a questionnnaire

TABLE I
Representative excerpt of heartbeat-data for
panelists that evaluated
Amarocite ® (1,1-dimethoxy-2,2,5-trimethyl-4-hexene)
tested as 1 wt.-% solution in mineral oil
Heartbeat (bpm)

| Panelist | Before | After | Difference |
|---|---|---|---|
| A | 84 | 90 | +6 |
| B | 68 | 77 | +9 |
| C | 56 | 81 | +25 |
| D | 64 | 70 | +6 |
| E | 68 | 78 | +10 |
| F | 60 | 72 | +12 |
| G | 59 | 71 | +12 |
| Mean | 65.6 | 77 | +11.4 |

After administering Amarocite® (1,1-dimethoxy-2,2,5-trimethyl-4-hexene) to the panelists according to the above protocol, an average heart rate increase of 17.3% was observed.

Very similar results were obtained with a solution of a mixture of 0.4 wt.-% of tetrahydrogeraniol and 0.4 wt.-% of aldehyde C10 (tested in mineral oil).

Similar results were obtained with a solution of a mixture of 0.3 wt.-% of I-menthol and 0.5 wt.-% of aldehyde C10 (tested in mineral oil).

Stronger energizing effects were observed with 0.8 wt.-% of tetrahydrogeraniol alone or with 2.0 wt.-% methyl dihydro jasmonate alone (both tested as solutions in mineral oil).

### Example 2

### Fragrances with Energizing Effect According to the Present Invention

| | Fragrance Type | | | | |
|---|---|---|---|---|---|
| Compound | Jasmine | White Tea | Mint | Red Grapefruit | Rose |
| AMAROCITE ® | | 3 | | 40 | |
| HEDIONE ® | 80 | 400 | | 10 | |
| MENTHOL L | | | 622 | | |
| TETRAHYDROGERANIOL | | | | | 300 |
| AGRUMEX ® | 68 | 9 | | | |
| AGRUNITRIL 10% in DPG | | 2 | | | |
| ALDEHYDE C8 | | | | 60 | |
| ALDEHYDE C8 10% in DPG | | 3 | | | |
| ALDEHYDE C9 | | | | 10 | |
| ALDEHYDE C10 | | | | 50 | |
| ALDEHYDE C10 10% in DPG | | 5 | | | |
| ALDEHYDE C11 | | | | 2 | 2 |
| UNDECYLENIC | | | | | |
| ALDEHYDE C12 LAURIC | | | | 4 | |
| ALDEHYDE C14 SO-CALLED | 8 | | | | |
| ALDEHYDE C14 SO-CALLED | | 5 | | | |
| 10% in DPG | | | | | |
| ALLYL AMYL GLYCOLATE | 2 | | | | |
| ALLYL CAPROATE | 12 | | | | |
| ALLYL CYCLOHEXYL | 10 | | | | |
| PROPIONATE | | | | | |
| ALLYL HEPTOATE | 32 | | | | |
| ALLYL IONONE | | 1 | | | |
| AMBROXAN ® 10% in DPG | | 4 | | | |
| ANETHOL | 2 | | 1 | | |
| ANISYL ACETATE | | | | 2 | |
| BENZALDEHYDE | | | 2 | | |
| BOURGEONAL ® | | | | 6 | |
| CARDAMOM OIL | | 1 | | | |
| CARVONE L | | | | 2 | |
| DAMASCENON MELANGE 1:1 | | | | | 1 |
| DAMASCENONE 10% in DPG | | 5 | | | |
| DAMASCONE DELTA 10% in | | 5 | | | |
| DPG | | | | | |
| DIHYDROEUGENOL | | | 1 | | 30 |
| DIHYDROMYRCENOL | 85 | | | | |
| DIMETHYL BENZYL | 10 | 5 | | | |
| CARBINYL ACETATE | | | | | |
| DIMETHYL BENZYL | 43 | 3 | | | |
| CARBINYL BUTYRAT | | | | | |
| DIPROPYLENE GLYCOL | | 175 | | 686 | |
| ETHYL BUTYRATE | 5 | | | | |
| ETHYL CAPROATE 10% in | | 4 | | | |
| DPG | | | | | |
| ETHYL LINALOOL | | | | 20 | |
| ETHYL MALTOL 1% in DPG | 2 | | | | |
| ETHYL MALTOL 5% in DPG | | | | 40 | |
| ETHYL METHYL BUTYRATE-2 | 15 | | | | |
| ETHYL VANILLIN | | | | 2 | |
| ETHYLENE BRASSYLATE | 10 | 40 | | | |
| EUCALYPTOL NAT. | | | 20 | | |
| EVERNYL ® 10% in DPG | | 4 | | | |
| FLORAZON ® | | 1 | | | |
| FREESIOL/CORPS 119 | | 15 | | | |
| GERANIOL | | 6 | | | |
| GERANIUM OIL | | | 1 | | |
| GINGER OIL | | 1 | | | |
| GLOBALIDE ® | | 20 | | | |
| GRAPEFRUIT BASE | | | | 10 | |
| HERBAFLORAT ® | 36 | | | | |
| HERBYL PROPIONATE | 23 | | | | |
| HEXENOL CIS-3 | | 2 | | 2 | |
| HEXENOL CIS-3 10% in DPG | 21 | | | | |
| HEXENYL ACETATE CIS-3 | 36 | | | | |
| 10% in DPG | | | | | |
| HEXYL ACETATE | 7 | | | | |
| INDOLE 10% in DPG | | 2 | | | |
| IONONE BETA | | 20 | | | |
| ISO E SUPER ® | | 30 | | | |
| JASMOPYRANE | 37 | | | | |
| LEMONGRASS OIL | | 3 | | | |
| LIGUSTRAL ® | | 4 | | | |
| LILIAL ® | | 15 | | | |
| LINALOOL OXIDE | | | | 4 | |
| LINALYL ACETATE | | 95 | | | |
| LINALYL ISOBUTYRATE | | | | 10 | |
| MACROLIDE ® 50% in TEC | | | | 6 | |
| MALTOL | | | 1 | | |
| MELONAL ® | 3 | | | | |
| MENTHONE L/ | | | | | 10 |
| ISOMENTHONE D ratio 82/18 | | | | | |
| MENTHONE/ISOMENTHONE | | | 300 | | |
| RAC. | | | | | |
| MENTHYL ACETATE RAC. | | | 30 | | |
| METHYL ANTHRANILATE 1% | | 10 | | | |
| in DPG | | | | | |
| METHYL HEPTENONE-6,5,2 | | | | 10 | |
| MUGETANOL ® | 35 | | | | |
| OCTINIA BASE 10% in DPG | 2 | | | | |
| ORANGE 20X BASE | | | 2 | | |
| ORANGE OIL 5X | | 5 | | | |
| ORANGENAL NAT. | | | | 4 | |
| OXANTHIA 50% in TEC 1% in | 6 | 5 | | | |
| DPG | | | | | |
| OXANTHIA 50% in TEC | | | | 6 | |
| PEACHOLIDE 10% in DPG | | 4 | | | |
| PHENIRAT ® | 76 | | | | |
| PHENOXANOL ® | | 30 | | | |
| PHENYLETHYL ACETATE | | | | | 15 |
| PHENYLETHYL ALCOHOL | | 15 | | | |
| PHENYLETHYL ALCOHOL | 77 | | | | 641 |
| PHENYLETHYL | | 3 | | | |
| PHENYLACETATE | | | | | |
| PRENYL ACETATE | 3 | | | | |
| ROSE OXIDE L | | | | | 1 |
| SPEARMINT OIL | | | 10 | | |
| TETRAHYDROLINALOOL | 90 | 30 | | | |
| TETRAHYDROMUGUOL | 33 | | | | |
| THYME OIL | | | 10 | | |
| THYMOL 10% in DPG | | 5 | | | |
| UNDECAVERTOL ® 10% in | 31 | | | | |
| DPG | | | | | |
| VANILLIN 10% in DPG | | | | 4 | |
| VETIKOL ACETATE 1% in | | 5 | | | |
| DPG | | | | | |
| TOTAL | 900 | 1,000 | 1,000 | 990 | 1,000 |

DPG = dipropylene glycol;
NAT. = natural;
RAC. = racemic;
TEC = triethyl citrate

### Example 3

### Personal Care Composition According to the Present Invention with an Energizing Sensory Complex

1 wt.-% of Fragrance Type “Jasmine” of example 2 was mixed with a cleansing surfactant composition. This cleaning surfactant composition contained sodium lauroamphodiacetate, sodium laureth-13 carboxylate, sodium trideceth sulfate, polysorbate-20, PEG-150 distearate, POE 80 sorbitan monolaurate, cocamidopropyl betaine, tetrasodium EDTA, quaternium 15, citric acid, sodium chloride, and water.

### Example 4

### Antiperspirant Stick According to the Present Invention with an Energizing Sensory Complex

| Part | % Wt | Ingredient | Supplier |
|---|---|---|---|
| 1 | 2.00 | Gelling Agent GP-1 | Ajinonoto |
| | | (Dibutyl Lauroyl Glutamide) | |
| 2 | 6.00 | Casid HSA | CasChem |
| | | (Hydroxystearic Acid) | |
| 3 | 48.05 | DC 244 Fluid | Dow Corning |
| | | (Cyclomethicone) | |
| 4 | 13.9 | Eutanol G | Cognis Care |
| | | (Octyldodecanol) | |
| 5 | 1.25 | Performathox 450 | New Phase Technologies |
| | | (C20-40 Pareth-10) | |
| 6 | 1.25 | Performathox 480 | New Phase Technologies |
| | | (C20-40 Pareth 40) | |
| 7 | 0.50 | Performacol 425 | New Phase Technologies |
| | | (C20-40 Alcohols) | |
| 8 | 0.05 | Versene NA | Dow Chemical |
| | | (Disodium EDTA) | |
| 9 | 25.0 | Reach AZP-908 SUF | Reheis |
| | | (Aluminum Zirconium | |
| | | Tetrachlorohydrex Gly) | |
| 10 | 0.75 | Fragrance Type “Jasmine” | Example 2 |

Compounding Procedure: add the ingredient parts 1-7 to a suitable vessel and heat to 80° C. Mix until all waxes are melted and clear homogeneous solution is achieved. Allow to cool to 70° C. with continued mixing. Add ingredients 8 and 9 with mixing until fully dispersed. Continue to mix while cooling to 65° C. Add ingredient 10 and mix and pour into stick containers.

### Example 5

### Antiperspirant Aerosol According to the Present Invention with an Energizing Sensory Complex

| Part | % Wt | Ingredient | Supplier |
|---|---|---|---|
| 1 | 10.00 | DC Fluid 244 | Dow Corning |
| | | (Cyclomethicone) | |
| 2 | 7.50 | Micro Dry | Reheis |
| | | (Aluminum Chlorohydrate) | |
| 3 | 1.25 | Myritol PC | Cognis Care |
| | | (Propylene Glycol Dicaprylate/Dicaprate) | |
| 4 | 0.75 | Fragrance Type “White Tea” or | Example 2 |
| | | Type “Rose” | |
| 5 | 80.0 | Hydrocarbon Propellant A-46 | |

Compounding Procedure: combine parts 1-4 in a suitable vessel. Mix until a homogeneous dispersion is achieved. Fill into aerosol cans, crimp on valve and pressure fill propellant (part 5) (a 20/80 blend of propane/isobutane).

### Example 6

### Antiperspirant Stick According to the Present Invention with an Energizing Sensory Complex

| | % by | % by |
|---|---|---|
| Ingredient | weight | weight |
| Phenyl Trimethicon (SilCare TM Silicone 15 M | 13.50 | 13.50 |
| 50) | | |
| Cetearyl alcohol | To 100 | To 100 |
| Cetiol CC (dicaprylyl carbonate) | 13.50 | 13.50 |
| Stearic acid | 3.50 | 3.50 |
| PEG-40-hydrated castor oil (Emulsogen TM | 4.10 | 4.10 |
| HCO 040) | | |
| PEG-8 distearate (Cithrol 4 DS) | 4.10 | 4.10 |
| Petrolatum | 6.90 | 6.90 |
| Aluminum hydrochlorate | 13.80 | 13.80 |
| Aluminum zirconium trichlorohydrex gly | 20.00 | 19.50 |
| Neo Heliopan ® Hydro (phenylbenzimidazole | 2.00 | 0.50 |
| sulphonic acid, Symrise) | | |
| 2,2-dimethyl-3-phenylpropanol | — | 0.25 |
| Ethylhexylglycerine (octoxyglycerin) | — | 0.30 |
| 1,1-dimethyl-3-phenylpropanol | — | 0.25 |
| Fragrance Type “Rose” of Example 2 | 0.60 | — |
| Fragrance Type “Red Grapefruit” of Example 2 | — | 0.75 |

### Example 7

### Suspension Roll-On According to the Present Invention with an Energizing Sensory Complex

| | % by | % by |
|---|---|---|
| Ingredient | weight | weight |
| Silicone | To 100 | To 100 |
| Ethylhexylglycerine (octoxyglycerin) | 1.00 | 1.00 |
| Quaternium-18 hectorite | 13.00 | 13.20 |
| Aluminum hydrochlorate, powder | 21.00 | 20.00 |
| 1,1-dimethyl-3-phenylpropanol | 0.25 | 0.50 |
| 4-methyl-4-phenyl-2-pentanol | 0.10 | — |
| Fragrance Type “White Tea” of Example 2 | 0.25 | — |
| Fragrance Type “Red Grapefruit” of Example 2 | — | 0.85 |
| Fragrance Type “Jasmine” of Example 2 | 0.50 | — |

### Example 8

### Suspension Stick According to the Present Invention with an Energizing Sensory Complex

| | % by | % by |
|---|---|---|
| Ingredient | weight | weight |
| Stearyl alcohol | 20.00 | 20.00 |
| Cyclomethicone | To 100 | To 100 |
| PPG-14 butyl ether | 2.00 | 2.00 |
| Hydrated castor oil | 1.00 | 1.00 |
| Talc | 2.00 | 2.00 |
| Aluminum hydrochlorate, powder | 20.00 | 20.00 |
| Triclosan ® (5-chloro-2-(2,4- | 0.30 | — |
| dichlorphenoxy)phenol) | | |
| Ethylhexylglycerine (octoxyglycerin) | 0.50 | 0.80 |
| 1,1-dimethyl-3-phenylpropanol | 0.30 | 0.40 |
| 2,2-dimethyl-3-phenylpropanol | 0.30 | 0.15 |
| Fragrance Type “White Tea” of Example 2 | 0.35 | — |
| Fragrance Type “Red Grapefruit” of Example 2 | 0.50 | — |
| Fragrance Type “Jasmine” of Example 2 | — | 0.60 |
| Fragrance Type “Mint” of Example 2 | — | 0.10 |

### Example 9

### Chewing Gum According to the Present Invention with an Energizing Sensory Complex

Chewing gum base K2 comprised 28.5% terpene resin, 33.9% polyvinyl acetate (MW=14,000), 16.25% hydrogenated plant oil, 5.5% mono- and diglycerides, 0.5% polyisobutene (MW 75,000), 2.0% butyl rubber (isobutene/isoprene copolymer), 4.6% amorphous silicon dioxide (water content approx. 2.5%), 0.05% antioxidant tert-butylhydroxytoluene (BHT), 0.2% lecithin, and 8.5% calcium carbonate. Chewing gum base K2 and the chewing gums can be prepared analogously to U.S. Pat. No. 6,986,907, incorporated herein in its entirety.

| | I (wt. %) | II (wt. %) |
|---|---|---|
| Chewing gum base K2 | 25.30 | 26.30 |
| Sorbitol | Ad 100 | Ad 100 |
| Glycerol | 2.40 | 2.40 |
| Lecithin | 7.00 | 7.00 |
| Aspartame | 0.14 | 0.14 |
| Encapsulated aspartame | 0.68 | 0.68 |
| Menthol, spray-dried | 0.25 | 0.50 |
| Lemon aroma, spray-dried | — | 0.20 |
| Fragrance Type “Mint” of | 1.25 | 0.95 |
| Example 2 | | |

The chewing gums of recipe (I) were shaped as strips, and those of recipe (II) were shaped as pellets.

### Example 10

### Non-Stick Chewing Gum According to the Present Invention with an Energizing Sensory Complex

Chewing gum base K1 comprised 2.0% butyl rubber (isobutene/isoprene copolymer, MW 400,000), 6.0% polyisobutene (MW=43,800), 43.5% polyvinyl acetate (MW 12,000), 31.5% polyvinyl acetate (MW=47,000), 6.75% triacetin and 10.25% calcium carbonate. Chewing gum base K1 and the chewing gums (I) and (II) can be prepared analogously to U.S. Pat. No. 5,601,858, incorporated herein in its entirety.

| | I | |
|---|---|---|
| | (wt. %) | II (wt. %) |
| Chewing gum base K1 | 26.00 | 26.00 |
| Triacetin | 0.25 | 0.25 |
| Lecithin | 0.50 | 0.50 |
| Sorbitol, crystalline | Ad 100 | Ad 100 |
| Mannitol | 15.30 | 15.20 |
| Glycerol | 12.10 | 12.00 |
| Aspartame | 0.17 | 0.17 |
| Encapsulated aspartame | 1.08 | 1.08 |
| Amorphous silica | 1.00 | 1.00 |
| Cottonseed oil | 0.50 | 0.50 |
| Polyoxyethylene sorbitan monolaurate (E-432) | 1.00 | 1.00 |
| Menthone glycerine acetal (Frescolat ® MGA) | — | 0.15 |
| Encapsulated spearmint flavor (contains I-carvone) | 0.20 | 0.10 |
| Encapsulated wintergreen flavor (contains methyl | — | 0.10 |
| salicylate) | | |
| Fragrance Type “Mint” of Example 2 | 1.40 | 1.00 |

The chewing gums of recipe (I) were shaped as strips, and those of recipe (II) were shaped as pellets.

### Example 11

### O/W Lotion According to the Present Invention with an Energizing Sensory Complex

| | % by | % by | % by |
|---|---|---|---|
| Components | weight | weight | weight |
| Paraffin oil | 5.00 | 5.00 | 5.00 |
| Isopropylpalmitate | 5.00 | 5.00 | 5.00 |
| Cetyl alcohol | 2.00 | 2.00 | 2.00 |
| Beeswax | 2.00 | 2.00 | 2.00 |
| Ceteareth-20 | 2.00 | 2.00 | 2.00 |
| PEG-20-glyceryl stearate | 1.50 | 1.50 | 1.50 |
| Glycerine | 3.00 | 3.00 | 1.50 |
| Phenoxy ethanol | 0.50 | 0.50 | — |
| 1,2-Pentanediol | — | — | 2.00 |
| Parabens (mixture of methyl-, ethyl-, | — | — | 0.30 |
| propyl-, butyl-, isobutylparaben) | | | |
| Fragrance Type “Jasmine” of Example 2 | 0.70 | — | — |
| Fragrance Type “White Tea” of Example 2 | — | 0.90 | — |
| Fragrance Type “Rose” of Example 2 | — | — | 0.80 |
| Water | To 100 | To 100 | To 100 |

### Example 12

### Hair Conditioner with UV Protection According to the Present Invention with an Energizing Sensory Complex

| | | % by | % |
|---|---|---|---|
| Components | INCI Name | weight | by weight |
| Renex PEG 6000 | PEG-150 | 2.50 | 2.50 |
| Hair Conditioner Base | Cetyl Alcohol, Behentrimonium | 3.00 | 3.00 |
| | Chloride, Triticum Vulgare | | |
| | (Wheat) Bran Extract, Linoleic Acid | | |
| PCL-Solid | Stearyl Heptanoate, Stearyl | 0.50 | 0.50 |
| | Caprylate | | |
| Dow Corning 5200 | Laurylmethicone Copolyol | 0.50 | 0.50 |
| Natrosol 250 HR | Hydroxyethylcellulose | 0.50 | 0.50 |
| Benzophenon-4 | Benzophenone-4 | 1.00 | 0.50 |
| Neo Heliopan AP | Disodium Phenyl | 1.00 | 0.80 |
| | Dibenzimidazole Tetrasulfonate | | |
| Amino Methyl propanol | Amino Methyl propanol | 2.00 | 1.20 |
| Nipagin M | Methylparaben | 0.30 | 0.30 |
| Dow Corning 949 Cationic | Amodimethicone, Cetrimonium | 2.00 | 2.00 |
| Emulsion | Chloride, Trideceth-12 | | |
| Fragrance Type “Jasmine” | Perfume | 0.55 | — |
| of Example 2 | | | |
| Fragrance Type “Rose” of | Perfume | — | 0.70 |
| Example 2 | | | |

### Example 13

### Mouthwash Concentrate According to the Present Invention with an Energizing Sensory Complex

| | I (wt. %) | II (wt. %) | III (wt. %) |
|---|---|---|---|
| Ethanol, 95% strength | 80.00 | 80.00 | 80.00 |
| Na cyclamate | 0.15 | 0.15 | 0.15 |
| Eucalyptol aroma (contains | — | — | 1.00 |
| natural eucalyptol) | | | |
| Dyestuff | 0.01 | 0.01 | 0.01 |
| Fragrance Type “Mint” of Example 2 | 3.50 | 1.00 | — |
| Fragrance Type “White Tea” of | — | 2.50 | 3.00 |
| Example 2 | | | |
| Dist. water | Ad 100.00 | Ad 100.00 | Ad 100.00 |

### Example 14

### Dental Cream According to the Present Invention with an Energizing Sensory Complex

| | I (wt. %) | II (wt. %) | III (wt. %) |
|---|---|---|---|
| Carrageenan | 0.90 | 0.90 | 0.90 |
| Glycerol | 15.00 | 15.00 | 15.00 |
| Sorbitol 70%, in water | 25.00 | 25.00 | 25.00 |
| PEG 1000 | 3.00 | 3.00 | 3.00 |
| Na-fluoride | 0.24 | 0.24 | 0.24 |
| Tetrapotassium diphosphate | 4.50 | 4.50 | 4.50 |
| Tetrasodium diphosphate | 1.50 | 1.50 | 1.50 |
| Na saccharinate | 0.40 | 0.40 | 0.40 |
| Precipitated silica | 20.00 | 20.00 | 20.00 |
| Titanium dioxide | 1.00 | 1.00 | 1.00 |
| p-Hydroxybenzoic acid methyl | 0.10 | 0.10 | 0.10 |
| ester | | | |
| Fragrance Type “Red Grapefruit” of | 1.00 | — | — |
| Example 2 | | | |
| Fragrance Type “White Tea” of | — | 1.25 | — |
| Example 2 | | | |
| Fragrance Type “Mint” of Example 2 | 0.50 | — | 1.30 |
| Sodium dodecyl sulfate | 1.30 | 1.30 | 1.30 |
| Dist. water | Ad 100.00 | Ad 100.00 | Ad 100.00 |

### Example 15

### Dental Cream According to the Present Invention with an Energizing Sensory Complex

| | I (wt. %) | II (wt. %) | III (wt. %) |
|---|---|---|---|
| Na-carboxymethylcellulose | 0.70 | 0.70 | 0.70 |
| Xanthan Gum | 0.50 | 0.50 | 0.50 |
| Glycerol | 15.00 | 15.00 | 15.00 |
| Sorbitol 70%, in water | 12.00 | 12.00 | 12.00 |
| K-nitrate | 5.00 | 5.00 | 5.00 |
| Na-monofluorophosphate | 0.80 | 0.80 | 0.80 |
| p-Hydroxybenzoic acid methyl | 0.15 | 0.15 | 0.15 |
| ester | | | |
| p-Hydroxybenzoic acid propyl | 0.05 | 0.05 | 0.05 |
| ester | | | |
| Na saccharinate | 0.20 | 0.20 | 0.20 |
| Wintergreen aroma (contains | 0.80 | — | 0.60 |
| methyl salicylate) | | | |
| Fragrance Type “Jasmine” of | 0.50 | — | — |
| Example 2 | | | |
| Fragrance Type “Mint” of Example 2 | — | 1.30 | 0.65 |
| Ca-carbonate | 35.00 | 35.00 | 35.00 |
| Silicon dioxide | 1.00 | 1.00 | 1.00 |
| Sodium dodecyl sulfate (SDS) | 1.50 | 1.50 | 1.50 |
| Dist. water | Ad 100.00 | Ad 100.00 | Ad 100.00 |

### Example 16

### Dental Gel According to the Present Invention with an Energizing Sensory Complex

| | I (wt. %) | II (wt. %) | III (wt. %) |
|---|---|---|---|
| Na-carboxymethylcellulose | 0.40 | 0.40 | 0.40 |
| Sorbitol 70%, in water | 72.00 | 72.00 | 72.00 |
| Polyethylene glycol (PEG) 1500 | 3.00 | 3.00 | 3.00 |
| Na-saccharinate | 0.07 | 0.07 | 0.07 |
| Na-fluoride | 0.24 | 0.24 | 0.24 |
| p-Hydroxybenzoic acid (PHB) ethyl | 0.15 | 0.15 | 0.15 |
| ester | | | |
| Abrasive silica | 11.00 | 11.00 | 11.00 |
| Thickening silica | 6.00 | 6.00 | 6.00 |
| Triclosan (2,4,4′-trichlor-2′- | 0.50 | 0.30 | 0.30 |
| hydroxydiphenyl ether) | | | |
| Cooling agent WS-3 | 0.10 | 0.08 | — |
| Fragrance Type “White Tea” of | 1.10 | — | — |
| Example 2 | | | |
| Fragrance Type “Mint” of Example 2 | — | 1.10 | 1.40 |
| Sodium dodecyl sulfate (SDS) | 1.40 | 1.40 | 1.40 |
| Dist. water | Ad 100.00 | Ad 100.00 | Ad 100.00 |

### Example 17

### Skin Oil According to the Present Invention with an Energizing Sensory Complex

| | I (wt. %) | II (wt. %) | III (wt. %) |
|---|---|---|---|
| Cetyl palmitate | 3.00 | 3.00 | 3.00 |
| C₁₂₋₁₅-Alkyl benzoate | 2.00 | 2.00 | 2.00 |
| Polyisobutene | 10.00 | 10.00 | 10.00 |
| Squalane | 2.00 | 2.00 | 2.00 |
| Fragrance Type “White Tea” of | 0.50 | — | — |
| Example 2 | | | |
| Fragrance Type “Jasmine” of | — | 0.50 | — |
| Example 2 | | | |
| Fragrance Type “Rose” of Example 2 | — | — | 0.50 |
| Preservative | q.s. | q.s. | q.s. |
| Mineral oil | ad 100 | ad 100 | ad 100 |

### Example 18

### Shampoo According to the Present Invention with an Energizing Sensory Complex

| | | | |
|---|---|---|---|
| Sodium laurylether sulfate (Texapon | 20.00% | 15.00% | 12.00% |
| NSO, Cognis) | | | |
| Cocamidopropyl betaine (Dehyton K, | — | 1.00% | 3.00% |
| Cognis) | | | |
| Sodium chloride | 1.40% | 1.40% | 1.40% |
| Citric acid | 1.30% | 1.30% | 1.30% |
| Phenoxyethanol | 0.30% | 0.30% | 0.30% |
| Methyl-, Ethyl-, Butyl-, and Propyl | 0.30% | 0.30% | 0.30% |
| parabenes | | | |
| Fragrance Type “White Tea” of | 0.50% | — | — |
| Example 2 | | | |
| Fragrance Type “Jasmine” of | — | 0.50% | — |
| Example 2 | | | |
| Fragrance Type “Rose” of Example 2 | — | — | 0.50% |
| Water | Ad 100 | Ad 100 | Ad 100 |

### Example 19

### Air Freshener Gel According to the Present Invention with an Energizing Sensory Complex

| | | I | | III |
|---|---|---|---|---|
| | | (wt. %) | II (wt. %) | (wt. %) |
| Water | | Ad 100 | Ad 100 | Ad 100 |
| Genugel ® X-6424 | Carrageenan | 2.00 | 2.00 | 2.00 |
| Arkopal ® N 100 | Emulsifier | 3.50 | 3.50 | 3.50 |
| Preventol ® D 7 | Preservative | 5.00 | 5.00 | 5.00 |
| Fragrance Type “Jasmine” | Perfume | 0.60 | — | — |
| of Example 2 | | | | |
| Fragrance Type “Red | Perfume | — | 1.00 | — |
| Grapefruit” of Example 2 | | | | |
| Fragrance Type “Rose” of | Perfume | — | — | 0.80 |
| Example 2 | | | | |

### Example 20

### Deodorant Sticks According to the Present Invention with an Energizing Sensory Complex

| | I (wt. %) | II (wt. %) | III (wt. %) |
|---|---|---|---|
| Sodium stearate | 8.00 | 8.00 | 8.00 |
| 1,2-Hexanediol | 0.50 | 0.25 | 0.30 |
| 1,2-Octanediol | — | 0.25 | 0.30 |
| 1,2-Propylene glycol | 41.00 | 41.00 | 41.00 |
| Butylene glycol | 5.00 | 5.00 | 5.00 |
| 2-Hexyldecanoic acid | 0.50 | — | 0.50 |
| 2-Butyoctanoic acid | — | 0.50 | — |
| Ceteareth-25 | 3.00 | 3.00 | 3.00 |
| Ethanol | 18.00 | 18.00 | 18.00 |
| Fragrance Type “White Tea” of | 0.25 | — | — |
| Example 2 | | | |
| Fragrance Type “Jasmine” of | — | 0.30 | — |
| Example 2 | | | |
| Fragrance Type “Rose” of Example 2 | — | — | 0.40 |
| Water | Ad 100 | Ad 100 | Ad 100 |

### Specific Embodiments

In a specific embodiment one, the invention is a method of energizing a human being comprising the following steps:
providing an effective amount of a fragrance material comprising or consisting of one or more compounds selected from group (a) consisting of:

(a) tetrahydrogeraniol, methyl dihydro jasmonate, I-menthol, 1,1-dimethoxy-2,2,5-trimethyl-4-hexene, n-decanal, diethyl malonate, and anethol, and
administering said effective amount of fragrance material to said human being in a way that said amount of fragrance material reaches the nasal cavity of said human being,

wherein said administered amount of fragrance material is capable of increasing the heart rate of said human being, preferably by 5% to 100%, more preferably by 5 to 50%, most preferably by 10 to 25%, based on the heart rate of said human being before administration of said amount of fragrance material.

In a specific embodiment two, the invention is a method according to specific embodiment one wherein said one or more compounds of group (a) are selected from the group consisting of:

tetrahydrogeraniol, methyl dihydro jasmonate, I-menthol, 1,1-dimethoxy-2,2,5-trimethyl-4-hexene, a mixture of n-decanal and tetrahydrogeraniol, a mixture of n-decanal and I-menthol, a mixture of I-menthol and diethyl malonate, a mixture of 1,1-dimethoxy-2,2,5-trimethyl-4-hexene and anethol.

In a specific embodiment three, the invention is a method according to specific embodiments one or two, wherein one or more compounds selected from said group (a), preferably said effective amount of said fragrance material comprising one or more compounds selected from said group (a), are part of a sensory complex, wherein the sensory complex comprises or consists of:

(i) an effective amount of a fragrance material comprising one or more compounds selected from the group (a) of the present invention, and additionally

(ii) one or more fragrance compounds different from the compounds of group (a),

(iii) one or more essential oils,

and/or

(iv) one or more cosmetically acceptable diluents.

In specific embodiment four, the invention is a method according to specific embodiment three wherein the sensory complex comprises or consists of:

(i) a total amount of from 1% to 80% by weight of one or more compounds selected from group (a) of the present invention, and additionally

(ii) a total amount of from 0.5% to 95% by weight of one of more fragrance compounds different from the compounds of group (a),

and/or

(iii) a total amount of from 0.01% to 30% by weight of one or more essential oils,

and/or

(iv) a total amount of from 1% to 80% by weight of one or more cosmetically acceptable diluents,

in each case based on the total weight of the sensory complex.

In a specific embodiment five, the invention is a method according to any preceding claim, wherein said effective amount of said fragrance material comprising one or more compounds selected from said group (a) is part of a personal care composition.

In a specific embodiment six, the invention is a method according to any of specific embodiments three or four, wherein said sensory complex is part of a personal care composition.

In a specific embodiment seven, the invention is a method according to specific embodiment five, wherein said effective amount of said fragrance material comprising one or more compounds selected from said group (a) is in the range of from 0.01 to 2%, preferably in the range of from 0.05 to 1%, based on the total weight of the personal care composition.

In a specific embodiment eight, the invention is a method according to specific embodiment six wherein the total amount of said sensory complex is in the range of from 0.05 to 10%, preferably in the range of from 0.2 to 2%, based on the total weight of the personal care composition.

In a specific embodiment nine, the invention is a method according to any of specific embodiments five to eight, wherein the personal care composition is selected from the group consisting of leave-on products, rinse-off products, oral care products, oral care devices, air-care products or air-care devices.

In a specific embodiment ten, the invention is a method according to specific embodiment nine, wherein the personal care composition is a leave-on product selected from the group consisting of wipes, gels, sticks, mousses, sprays, lotions, creams, facial masks, cleansing compositions, powders, and oils.

In a specific embodiment eleven, the invention is a method according to specific embodiment nine, wherein the personal care composition is a rinse-off product selected from the group consisting of wipes, washes, baths, shampoos, gels, soaps, sticks, balms, sachets, pillows, mousses, sprays, lotions, creams, cleansing compositions, powders, oils, and bath oils.

In a specific embodiment twelve, the invention is a method according to specific embodiment nine, wherein the personal care composition is an air-care product or device selected from the group consisting of pump-sprays, aerosols, candles, vaporizers, diffusers, wicking systems, fans, piezoelectric systems, heated oil baths, and incense sticks.

In a specific embodiment thirteen, the invention is a method according to specific embodiment nine, wherein the personal care composition is an oral care product or device selected from the group consisting of toothpastes, dentifrices, mouth sprays, mouth washes, chewing gums, mouth cleansers, and dental devices.

In a specific embodiment fourteen, the invention is use of a fragrance material comprising one or more compounds selected from the group (a) consisting of tetrahydrogeraniol, methyl dihydro jasmonate, I-menthol, 1,1-dimethoxy-2,2,5-trimethyl-4-hexene, n-decanal, diethyl malonate, and anethol, for energizing a human being.

What is claimed is:

1. A method of energizing a human being comprising the following steps:
providing an effective amount of a fragrance material comprising or consisting of one or more compounds selected from group (a) consisting of:
(a) tetrahydrogeraniol, methyl dihydro jasmonate, I-menthol, 1,1-dimethoxy-2,2,5-trimethyl-4-hexene, n-decanal, diethyl malonate, and anethol,
and
administering said effective amount of fragrance material to said human being in a way that said amount of fragrance material reaches the nasal cavity of said human being,
wherein said administered amount of fragrance material is capable of increasing the heart rate of said human being, based on the heart rate of said human being before administration of said amount of fragrance material.

2. A method according to claim 1 wherein said one or more compounds of group (a) are selected from the group consisting of:
tetrahydrogeraniol, methyl dihydro jasmonate, I-menthol, 1,1-dimethoxy-2,2,5-trimethyl-4-hexene, a mixture of n-decanal and tetrahydrogeraniol, a mixture of n-decanal and I-menthol, a mixture of I-menthol and diethyl malonate, a mixture of 1,1-dimethoxy-2,2,5-trimethyl-4-hexene and anethol.

3. A method according to claim 1, wherein one or more compounds selected from said group (a) are part of a sensory complex, wherein the sensory complex comprises or consists of:
(i) an effective amount of a fragrance material comprising one or more compounds selected from the group (a) of the present invention, and additionally
(ii) one or more fragrance compounds different from the compounds of group (a),
(iii) one or more essential oils,
and/or
(iv) one or more cosmetically acceptable diluents.

4. A method according to claim 3 wherein the sensory complex comprises or consists of:
(i) a total amount of from 1% to 80% by weight of one or more compounds selected from group (a) of the present invention, and additionally
(ii) a total amount of from 0.5% to 95% by weight of one of more fragrance compounds different from the compounds of group (a),
and/or
(iii) a total amount of from 0.01% to 30% by weight of one or more essential oils,
and/or
(iv) a total amount of from 1% to 80% by weight of one or more cosmetically acceptable diluents,
in each case based on the total weight of the sensory complex.

5. A method according to claim 1, wherein said effective amount of said fragrance material comprising one or more compounds selected from said group (a) is part of a personal care composition.

6. A method according to claim 3, wherein said sensory complex is part of a personal care composition.

7. A method according to claim 5, wherein said effective amount of said fragrance material comprising one or more compounds selected from said group (a) is in the range of from 0.01 to 2%, based on the total weight of the personal care composition.

8. A method according to claim 6 wherein the total amount of said sensory complex is in the range of from 0.05 to 10%, based on the total weight of the personal care composition.

9. A method according to claim 5, wherein the personal care composition is selected from the group consisting of leave-on products, rinse-off products, oral care products, oral care devices, air-care products or air-care devices.

10. A method according to claim 9, wherein the personal care composition is a leave-on product selected from the group consisting of wipes, gels, sticks, mousses, sprays, lotions, creams, facial masks, cleansing compositions, powders, and oils.

11. A method according to claim 9, wherein the personal care composition is a rinse-off product selected from the group consisting of wipes, washes, baths, shampoos, gels, soaps, sticks, balms, sachets, pillows, mousses, sprays, lotions, creams, cleansing compositions, powders, oils, and bath oils.

12. A method according to claim 9, wherein the personal care composition is an air-care product or device selected from the group consisting of pump-sprays, aerosols, candles, vaporizers, diffusers, wicking systems, fans, piezoelectric systems, heated oil baths, and incense sticks.

13. A method according to claim 9, wherein the personal care composition is an oral care product or device selected from the group consisting of toothpastes, dentifrices, mouth sprays, mouth washes, chewing gums, mouth cleansers, and dental devices.

14. A method for energizing a human being comprising administering to a human being a fragrance material comprising one or more compounds selected from the group (a) consisting of tetrahydrogeraniol, methyl dihydro jasmonate, I-menthol, 1,1-dimethoxy-2,2,5-trimethyl-4-hexene, n-decanal, diethyl malonate, and anethol.
